(19) United States
(12) Patent Application Publication
(10) Pub. No.: US 2005/0249823 A1
(43) Pub. Date: Nov. 10, 2005

(54) Methods for the prevention or amelioration of neuropsychiatric and related diseases

(76) Inventors: Tanya Kaye Murphy, Gainesville, FL (US); Nathan Andrew Shapira, Gainesville, FL (US); Mary Catherine Lessig, Seattle, WA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/975,701

(22) Filed: Oct. 28, 2004

Related U.S. Application Data
(60) Provisional application No. 60/517,350, filed on Nov. 4, 2003.

(51) Int. Cl. A61K 33/08; A61K 31/525; A61K 31/355; A61K 31/225

(52) U.S. Cl. 424/692; 514/251; 514/458; 514/356; 514/560; 514/547

(57) ABSTRACT

The present invention pertains to compositions and methods for therapeutically and/or prophylactically treating patients with neurological, neurogenetic, or psychiatric diseases, disorders, conditions, or distress. Specifically, the present invention relates to the administration of compositions containing vitamins B₆ and E, magnesium oxide, essential fatty acids, and folate.

### CROSS-REFERENCE TO RELATED APPLICATION

This application claims the benefit of provisional patent application Ser. No. 60/517,350, filed Nov. 4, 2003, which is hereby incorporated by reference in its entirety.

### BACKGROUND OF THE INVENTION

Vitamins play a vital role in fundamental biological pathways in tissues. Vitamins are particularly important in brain tissue due to its high metabolic rate and dependence on continuous metabolism. In fact, vitamins are closely linked to the sensitivity of the brain to deficiency (McIlwain, H. and Bachelard, H. S., “Nutritional factors and the central nervous system,” in Biochemistry and the Central Nervous System, 5th ed. London: Churchill Livingstone, 1985, pp. 244-281). Further, certain vitamin-dependent pathways in the brain are linked to neurotransmitter synthesis (Gibson, G. E. and Blass, J. P., “Nutrition and Brain Function,” in Basic Neurochemistry: Molecular, Cellular and Medical Aspects, 6^{th} ed. Philadelphia, Pa.: Lippincott-Raven, 1999).

For example, vitamin B₆ (also known as pyridoxine) is necessary for the biosynthesis of several neurotransmitters. The concentration of vitamin B₆ in the brain is normally about 100-fold higher than that in the blood. In vitamin B₆ deficient rats, biochemical and morphological abnormalities, including decreased dendritic arborization and reduced numbers of myelinated axons and synapses, are associated with behavioral changes, such as epileptic form of seizures and movement disorders. Reduced seizure threshold and delayed neuronal recovery are related to significantly reduced brain regional GABA and elevated glutamate concentrations in pyridoxine-deficient rats. (Sharma, S. K. et al., “Picrotoxin and pentylene tetrazole induced seizure activity in pyridoxine-deficient rats,” J. Neurol. Sci., 121:1-9 (1994)). In addition, vitamin B₆ deficiency during gestation and lactation alters the function of N-methyl-d-aspartate (NMDA) receptors, which are receptors for the neurotransmitter glutamate that is the most important excitatory transmitter in the brain.

It has been suggested that mild forms of pyridoxine dependence may be a relatively common cause of intractable seizures and mental retardation. Pyridoxine deficiency has occurred in human infants fed a formula from which vitamin B₆ had been inadvertently omitted. The prominent finding was that patients suffering from intractable seizures respond promptly to injections of the vitamin B₆. Deficiency of pyridoxine can contribute to the polyneuropathy of B-complex deficiency. In homocystinuria and cystothioninuria, two disorders of amino acid metabolism, some patients respond to large doses of pyridoxine. In these patients, the mutations appear to reduce the affinity of the relevant enzymes for pyridoxal phosphate (see Yudkoff, M., “Diseases of Amino Acid Metabolism,” in Basic Neurochemistry: Molecular, Cellular and Medical Aspects, 6^{th} ed. Philadelphia, Pa.: Lippincott-Raven, 1999).

Further, hydrazides and oximes can increase pyridoxine requirements. For example, large doses of pyridoxine are routinely given with the antituberculous medication isonicotinic hydrazide, to prevent drug-induced neuropathy.

Vitamin E (also known as tocopherol) is one of the main antioxidants in the brain (Gibson, G. E. and Blass, J. P., “Nutrition and Brain Function,” in Basic Neurochemistry: Molecular, Cellular and Medical Aspects, 6^{th} ed. Philadelphia, Pa.: Lippincott-Raven, 1999), levels of which can be manipulated by diet. Dietary manipulation of antioxidants has practical implications for brain function. For example, age-associated free radical damage in the brain (see Dugan, L. L. and Choi, D. W., “Hypoxic-Ischemic Brain Injury and Oxidative Stress,” in Basic Neurochemistry: Molecular, Cellular and Medical Aspects, 6^{th} ed. Philadelphia, Pa.: Lippincott-Raven, 1999) may be treated or prevented with the administration of certain antioxidants.

It is known that vitamin E deficiency produces a characteristic neurological syndrome, which presumably results from increased oxidative stress arising from a reduction in antioxidant capacity. Within the brain, the cortex, striatum and cerebellum are the most sensitive regions to vitamin E deficiency. Vitamin E deficiency in neural tissues increases endogenous lipid peroxidation, as evidenced in brain tissues by the appearance of thiobarbituric acid-reactive substances such as malondialdehyde. Several neurodegenerative disorders are associated with oxidative stress that is manifested by lipid peroxidation, protein oxidation and other markers. Included in these disorders in which oxidative stress is thought to play an important role in their pathogenesis are Alzheimer's disease (AD), Parkinson's disease (PD), amyotrophic lateral sclerosis (ALS), tardive dyskinesia, Huntington's disease (HD), and multiple sclerosis.

For example, vitamin E deficiency reduces tyrosine hydroxylase-immunopositive neurons in the substantia nigra. It has been noted that the enhanced sensitivity of the nigrostriatal pathway to oxidative stress could have important implications for the pathogenesis of Parkinson's disease (see Sian, J. et al., “Neurotransmitters and Disorders of the Basal Ganglia,” in Basic Neurochemistry: Molecular, Cellular and Medical Aspects, 6^{th} ed. Philadelphia, Pa.: Lippincott-Raven, 1999). A diet deficient in vitamin E increases glutamate and GABA and decreases tryptophan concentrations in the substantia nigra. The increase of nigral glutamate suggests possible links to degenerative processes through glutamatergic excitotoxicity. These results suggest that vitamin E may play a significant role in the degeneration of the substantia nigra and that this tissue may be particularly sensitive to oxidative stress.

Folic acid plays a key role in the transfer of one-carbon (active methylene) groups, including the conversion of serine to glycine and the cobalamin-dependent transfer from homocysteine to methionine. Although dietary deficiency of folate leads to anemia without significant neurological signs, both genetic and environmental disorders of folate metabolism have been associated with disease of the nervous system (see Yudkoff, M., “Diseases of Amino Acid Metabolism,” in Basic Neurochemistry: Molecular, Cellular and Medical Aspects, 6^{th} ed. Philadelphia, Pa.: Lippincott-Raven, 1999). Certain common medications, including phenyloin and certain antitumor agents, can increase requirements for dietary folate. Treatment with folate can mask the hematological signs of cobalamin deficiency without affecting the progressive damage to the nervous system.

Essential fatty acids are believed to play an important role in maintaining cardiac health, and more recently, evidence suggests a positive impact on mental health. The use of essential fatty acids (EFA), for different psychiatric disorders, ranging from schizophrenia and tardive dyskinesia to mood disturbances, has been documented in the literature. Childhood psychiatric disorders are believed to have multifactorial etiologies including stress, neurotransmitter abnormalities, and immune dysfunction. Early evidence points to n-3 EFA supplementation having an impact in all three of these areas. Overlap and interaction in neuromodulation and immunomodulation is possible and likely. Alterations in immune indices after EFA therapy have been mixed and not measured in children. Studies to date in children have focused primarily on chronic, relatively static psychiatric illness such as ADHD and autism. Moreover, the incidence and treatment of stroke in adults may be mediated via EFA, such as DHA, administration.

Current therapy for treating stroke is limited to thrombolysis, which has a narrow therapeutic window and requires sophisticated pretreatment imaging (“Tissue plasminogen activator for acute ischemic stroke: The National Institute of Neurological Disorders and Stroke rt-PA Stroke Study Group,” N. Engl. J. Med., 333:1581-1587 (1995); Chiu, D. et al., “Intravenous tissue plasminogen activator for acute ischemic stroke: feasibility, safety, and efficacy in the first year of clinical practice,” Stroke, 29:18-22 (1998)). The only Food and Drug Administration-approved therapy for acute ischemic stroke is i.v. recombinant tissue plasminogen activator (rtPA), which is indicated for selected patients who can be treated within 3 hours of the onset of a stroke. The use of rtPA is associated with an increased risk of intracerebral hemorrhage (ICH) and mortality (Katzan, I. L., et al., “Use of tissue-type plasminogen activator for acute ischemic stroke: the Cleveland area experience,” J. Am. Med. Assoc., 283:1151-1158 (2000)). Largely because of its limitations and risks, rtPA is administered to only a small fraction of all eligible patients (Katzan, I. L., et al., “Use of tissue-type plasminogen activator for acute ischemic stroke: the Cleveland area experience,” J. Am. Med. Assoc., 283:1151-1158 (2000); Albers, G. W. et al., “Intravenous tissue-type plasminogen activator for treatment of acute stroke: the Standard Treatment with Alteplase to Reverse,” J. Am. Med. Assoc., 283:1145-1150 (2000)).

Recently, the rapid transport of DHA across the BBB and its retention in the brain as ascorbic acid (AA) was described in rodents (Agus, D. B. et al., “Vitamin C crosses the blood-brain barrier in the oxidized form through the glucose transporters,” J. Clin. Invest., 100:2842-2848 (1997)). DHA has been examined previously as the product of reversible AA oxidation. Early studies demonstrated that DHA is antiscorbutic when given orally, suggesting a metabolic conversion to AA in vivo (Deutsch, J. C., “Dehydroascorbic acid,” J. Chromatogr., 881:299-302 (2000)). Because of its unique permeability properties at the BBB, DHA has been evaluated for potentially beneficial effects in conditions associated with antioxidant deficiency in the brain.

DHA has been shown, when administered intravenously in the setting of murine cerebral ischemia, to significantly improve cerebral blood flow and functional outcome and to significantly decrease the volume of infarcted brain tissue. The level of cerebroprotection achieved with DHA, not seen with AA, supports the hypothesis that the use of a potent antioxidant precursor, with clearly defined BBB penetrability, has promise in the treatment of thromboembolic stroke in humans.

However, there are no supplemental formulations currently available that provide both pediatric and adult patients with the appropriate amounts and proportions of vitamins, minerals and EFA to optimize neurological health. Further, traditional pharmaceutical agents used to treat neurological, neurogenetic, or psychiatric illnesses have not been particularly effective. Often, traditional methods of therapy are used with reservation, especially in pediatric and special needs populations. Natural methods are often as effective as manufactured treatments and are perceived as safer alternatives to current pharmaceutical agents. Natural supplements also often have minimized side effect profile as compared to their manufactured counterparts. Therefore, there remains a need for a nutritional formulation which can prevent or ameliorate symptomology associated with neurological, neurogenetic, or psychiatric disease, disorders, conditions, or distress.

### BRIEF SUMMARY OF THE INVENTION

The compositions of the present invention overcome the deficiencies of currently-available medications and/or supplements by providing formulations which are specifically tailored for pediatric or adult patients to optimize mental health. In particular, the present invention provides formulations for nutritional supplements that are specifically tailored to treat and/or prevent neurological, neurogenetic, or psychiatric diseases, disorders, conditions, or distress in both adult and pediatric patients. The compositions of the invention contain a novel combination of essential fatty acids and various vitamins and minerals.

In one embodiment, the present invention provides compositions containing vitamins B₆ and E, magnesium oxide, Essential Fatty Acids (also referred to herein as EFA), and folate. Contemplated dosage ranges for compositions of the invention include the following: in pediatric use, vitamin B₆ at a range of 5-15 mg, vitamin E at a range of 150-250 Iu, magnesium oxide at a range of 150-250 mg, Essential Fatty Acids at ranges of 600-1000 mg EPA and 150-250 mg DHA, and a folate at a range of 350-450 μg; in adult use B₆ at a range of 10-30 mg, vitamin E at a range of 300-500 Iu, magnesium oxide at a range of 300-500 mg, Essential Fatty Acids at ranges of 1200-2000 mg EPA and 300-500 DHA, and folate at a range of 700-1500 μg.

### DETAILED DISCLOSURE OF THE INVENTION

The present invention utilizes new oral formulations combining vitamins B₆ and E, magnesium oxide, Essential Fatty Acids, and folate. The formulations of the present invention are for use in treating and/or preventing neurological, neurogenetic, or psychiatric diseases, disorders, conditions, or distress in both adult and pediatric patients.

The term “patient,” as used herein, describes an organism, including mammals, to which treatment with the compositions according to the present invention is provided. Mammalian species that benefit from the disclosed methods of treatment include, but are not limited to, apes, chimpanzees, orangutans, humans, monkeys; and domesticated animals (i.e., pets) such as dogs, cats, mice, rats, guinea pigs, and hamsters.

The term “formulations,” in accordance with the present invention, refers to enteral (i.e., oral, sublingual, or rectal), parenteral, or topical (i.e., transdermal) forms. Organic or inorganic substances which do not reactive with the active ingredients can be used as supports (i.e., water, oil, benzyl alcohol, polyethylene glycol, glycerol triacetate, or other fatty acid glycerides, gelatine, lecithin, cyclodextrin, carbohydrates such as lactobiose or starch, magnesium stearate, talc, or cellulose).

The term “active substance,” as used herein, refers to substances comprising the combination of vitamins B₆ and E, magnesium oxide, EFA, and folate for the treatment, prevention, cure or mitigation of neurological, neurogenetic, or psychiatric diseases, disorders, conditions, or distress.

The term “biologically-acceptable,” as used herein, refers to any substance or substances that is safe for human consumption.

The term “effective amount,” as used herein, refers to the amount necessary to elicit the desired biological response. In accordance with the subject invention, the effective amount of a composition of the invention is the amount necessary to treat/prevent neurological, neurogenetic, or psychiatric diseases, disorders, conditions, or distress in adult or pediatric patients.

In one embodiment, the present invention provides compositions containing vitamins B₆ and E, magnesium oxide, Essential Fatty Acids, and folate. Contemplated dosage ranges for compositions of the invention include the following: in pediatric use, an effective amount of vitamin B₆ includes a range of 5-15 mg, an effective amount of vitamin E includes a range of 150-250 Iu, an effective amount of magnesium oxide includes a range of 150-250 mg, an effective amount of Essential Fatty Acids includes a range of 600-1000 mg eicosapentaenoic acid (EPA) and a range of 150-250 mg docosahexaenoic acid (DHA), and an effective amount of folate includes a range of 350-450 μg; in adult use an effective amount of vitamin B₆ includes a range of 10-30 mg, an effective amount of vitamin E includes a range of 300-500 Iu, an effective amount of magnesium oxide includes a range of 300-500 mg, an effective amount of Essential Fatty Acids includes a range of 1200-2000 mg EPA and a range of 300-500 DHA, and an effective amount of folate includes a range of 700-1500 μg.

The fatty acids of the present invention can be from any source including, without limitation, natural or synthetic oils, fats, waxes, or combinations thereof. Moreover, the fatty acids herein may be derived without limitation, from non-hydrogenated oils, partially hydrogenated oils, fully hydrogenated oils, or combinations thereof. Non-limiting exemplary sources of fatty acids include, flaxseed and/or other seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, evening primrose oil, jojoba, tallow, beef tallow, butter, chicken fat, lard, dairy butterfat, shea butter, or combinations thereof. Specific non-limiting exemplary fish or marine oil sources include shellfish oil, tuna oil, mackerel oil, salmon oil, menhaden, anchovy, herring, trout, sardines, or combinations thereof. Preferably, the source of the fatty acids of the invention is fish or marine oil, or flaxseed oil.

The present formulation may optionally contain additional vitamins, minerals, and/or EFA. Non-limiting exemplary vitamins, minerals, and EFA, and their derivatives thereof, for inclusion in the present formulations include, vitamins A, the remaining B vitamins (such as thiamine (B1), riboflavin (B2), niacin (B3), pyridoxine (B6), folic acid (B9), cyanocobalamin (B 12), pantothenic acid and biotin), C, D, K, iron, calcium, potassium, copper, chromium, zinc, molybdenum, iodine, boron, selenium, and manganese, and alpha-linolenic acid (ALA).

Various additives may be incorporated into the present formulations. Optional additives of the present formulations include, without limitation, starches, sugars, fats, antioxidants, amino acids, proteins, derivatives thereof or combinations thereof.

It is also possible in the formulations of the present invention to include various forms of release, which include and are not limited to, immediate release, extended release, pulse release, variable release, controlled release, timed release, sustained release, delayed release, long acting, and combinations thereon. The ability to obtain immediate release, extended release, pulse release, variable release, controlled release, timed release, sustained release, delayed release, long acting characteristics, and combinations thereof, is performed using well-known procedures and techniques available to the ordinary skilled artisan.

Biologically-acceptable formulations, in accordance with the present invention, can be administered to a patient in various forms. Examples of such forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, foods, nutritive foods, functional foods, and combinations thereof. The preparation of the above forms are well known to persons of ordinary skill in the art.

For example, quick dissolve tablets may be prepared by mixing the formulation of the present invention with agents such as sugars and cellulose derivatives, which promote dissolution or disintegration of the resultant tablet after oral administration, usually within 30 seconds.

Cereal coatings may be prepared, for example, by passing cereal (after it has been formed into pellets, flakes, or other geometric shapes for biologically acceptable ingestion) under a precision spray coating device to deposit a film of formulations of the present invention, plus excipients onto the surface of the formed cereals. The units thus treated are then dried to form a cereal coating.

By way of example, health bars can be prepared by mixing the formulations of the present invention with excipients (i.e., binders, fillers, flavors, colors, etc.) to a plastic mass consistency. The mass is then either extended or molded to form “health bar” shapes that are then dried or allowed to solidify to form the final product.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin based film using technology and machinery known to those in the soft gel industry. The industrial units so formed are then dried to constant weight.

Chewable tablets, for example, without limitation, may be prepared by mixing the formulations of the present invention with excipients designed to form a relatively soft, flavored, tablet dosage form that is intended to be chewed rather than swallowed. Conventional tablet machinery and procedures, that is both direct compression and granulation (i.e., or slugging, before compression) can be utilized. Those individuals involved in pharmaceutical solid dosage form production are well versed in the processes and the machinery used as the chewable dosage form is a very common dosage form in the pharmaceutical industry. Further, film coated tablets, for example, can be prepared by coating tablets using techniques such as rotating pan coating methods or air suspension methods to deposit a contiguous film layer on a tablet.

Compressed tablets, for example, may be prepared by mixing the formulation of the present invention with excipients intended to add binding qualities to disintegration qualities. The mixture is either directly compressed or granulated then compressed using methods and machinery quite well known to those in the industry. The resultant compressed tablet dosage units are then packaged according to market need (i.e., unit dose, rolls, bulk bottles, blister packs, etc.).

It is contemplated that formulations of the present invention can be combined with biologically-acceptable carriers which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders well known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

Plasticizers that can be used in combination with the formulations of the invention include, and are not limited to, have previously been dissolved in an organic solvent and are added in solution form. Preferred plasticizers may be selected from the group consisting of diethyl phthalate, diethyl sebacate, triethyl citrate, cronotic acid, propylene glycol, butyl phthalate, dibutyl sebacate, caster oil and mixtures thereof, without limitation. As is evident, the plasticizers of the invention may be hydrophobic as well as hydrophilic in nature. Water-insoluble hydrophobic substances, such as diethyl phthalate, diethyl sebacate and caster oil are used to delay the release of water-soluble vitamins, such as vitamin C. In contrast, hydrophilic plasticizers are used when water-insoluble vitamins are employed which aid in dissolving the encapsulated film, making channels in the surface, which aid in nutritional composition release.

Specific pediatric conditions that can be prevented and/or treated by administering the formulations of the present invention include, and are not limited to: anxiety disorders, including obsessive-compulsive disorder (OCD); learning disabilities and school problems (i.e., attention disorders, including attention deficit/hyperactivity disorder (ADHD)); tick disorders, including Tourettes Syndrome; autism; brain and spinal cord tumors; cerebral palsy; depression; epilepsy and seizure disorders; headaches; hydrocephalus; learning disorders; muscular dystrophy; multiple sclerosis; myasthenia gravis; neurofibromatosis and other neurocutaneous diseases; neurogenetic diseases; neuroimmune diseases; neurovascular disorders; scoliosis; sleep disorders; spasticity; spina bifida and myelodysplasia; spine anomalies; traumatic brain injury; tuberous sclerosis; neuromuscular diseases, metabolic degenerative diseases of the nervous system, developmental disorders of the nervous system; and conduct disorders.

Further, adult conditions that can be prevented and/or treated using the formulations of the present invention include, and are not limited to: amyotrophic lateral sclerosis (ALS); anxiety disorders, including obsessive-compulsive disorder (OCD); agnosia; Alzheimer's disease; Lou Gehrig's disease; Bell's Palsy; benign essential blepharospasm; cerebral palsy; chronic inflammatory demyelinating polyneuropathy; dementia; depression; epilepsy; headache; Huntington's disease; learning disabilities; migraine, multiple sclerosis; psychiatric disorders (i.e., schizophrenia, bipolar disorder, etc.); narcolepsy; motor neuron diseases; neurofibromatosis; Parkinson's disease; peripheral neuropathy; seizure disorder; stroke; tardive dyskinesia; tremor; and post traumatic stress disorder.

The dosage of the formulations of the present invention depend on the form of therapy, on the form of application of the formulation, and on the age, weight, nutrition, and condition of the patient. Treatment may be commenced with the dosage amounts provided above and can be increased until the optimum effect is achieved.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A composition consisting essentially of:
a) vitamin B₆;
b) vitamin E;
c) magnesium oxide;
d) essential fatty acids; and
e) folate.

2. The composition according to claim 1, wherein the essential fatty acids comprise eicosapentaenoic acid and docosahexaenoic acid.

3. The composition according to claim 2, wherein the vitamin B₆ is at a range of 5-15 mg, wherein the vitamin E is at a range of 150-250 Iu, wherein the magnesium oxide is at a range of 150-250 mg, the eicosapentaenoic acid is at a range of 600-1000 mg, wherein the docosahexaenoic acid is at a range of 150-250 mg, and the folate is at a range of 350-450 μg.

4. The composition according to claim 2, wherein the vitamin B₆ is at a range of 10-30 mg, wherein the vitamin E is at a range of 300-500 Iu, wherein the magnesium oxide is at a range of 300-500 mg, the eicosapentaenoic acid is at a range of 1200-2000 mg, wherein the docosahexaenoic acid is at a range of 300-500 mg, and the folate is at a range of 700-1500 μg.

5. The composition according to claim 1, wherein the essential fatty acids are oils selected from the group consisting of flaxseed oil, marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, evening primrose oil, jojoba, tallow, beef tallow, butter, chicken fat, lard, dairy butterfat, and shea butter.

6. The composition according to claim 5, wherein the marine oil is selected from the group consisting of shellfish oil, tuna oil, mackerel oil, salmon oil, menhaden oil, anchovy oil, herring oil, trout oil, and sardine oil.

7. The composition according to claim 1, further comprising an item selected from the group consisting of: vitamin A, thiamine (B1), riboflavin (B2), niacin (B3), pyridoxine (B6), folic acid (B9), cyanocobalamin (B12), pantothenic acid; biotin, vitamin C, vitamin D, vitamin K, iron, calcium, potassium, copper, chromium, zinc, molybdenum, iodine, boron, selenium, manganese, and alpha-linolenic acid (ALA).

8. The composition according to claim 1, further comprising additives.

9. The composition according to claim 8, wherein the additive is selected from the group consisting of starches, sugars, fats, antioxidants, amino acids, and proteins.

10. The composition according to claim 1, further comprising supports.

11. The composition according to claim 10, wherein the supports are selected from the group consisting of water, oil, benzyl alcohol, polyethylene glycol, glycerol triacetate, fatty acid glycerides, gelatine, lecithin, cyclodextrin, starch, magnesium stearate, and talc.

13. The composition according to claim 1, wherein the composition is provided in a biologically-acceptable formulation selected from the group consisting of: immediate release formulation, extended release formulation, pulse release formulation, variable release formulation, controlled release formulation, time release formulation, sustained release formulation, delayed release formulation, and long acting formulation.

14. The composition according to claim 13, wherein the formulation is provided in a form selected from the group consisting of chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, cereals, cereal coatings, nutritive foods, and functional foods.

15. The composition according to claim 14, wherein the formulation is provided in cereal.

16. The composition according to claim 14, wherein the formulation is provided in a health bar.

17. The composition according to claim 14, wherein the formulation is provided in a gelatin capsule.

18. The composition according to claim 14, wherein the formulation is provided in a tablet.

19. A method for treating or preventing a condition selected from the group consisting of neurological diseases; neurological disorders; neurological conditions; neurological distress; neurogenetic diseases; neurogenetic disorders; neurogenetic conditions; neurogenetic distress; psychiatric diseases; psychiatric disorders; psychiatric conditions; and psychiatric distress, said method comprising administering to a patient an effective amount of a composition consisting essentially of vitamin B₆, vitamin E, magnesium oxide, essential fatty acids, and folate.

20. The method according to claim 19, wherein the essential fatty acids comprise eicosapentaenoic acid and docosahexaenoic acid.

21. The method according to claim 20, wherein the effective amount of composition comprises vitamin B₆ at a range of 5-15 mg, vitamin E at a range of 150-250 Iu, magnesium oxide at a range of 150-250 mg, eicosapentaenoic acid at a range of 600-1000 mg, docosahexaenoic acid at a range of 150-250 mg, and folate at a range of 350-450 μg.

22. The method according to claim 20, wherein the effective amount of composition comprises vitamin B₆ at a range of 10-30 mg, vitamin E at a range of 300-500 Iu, magnesium oxide at a range of 300-500 mg, eicosapentaenoic acid at a range of 1200-2000 mg, docosahexaenoic acid at a range of 300-500 mg, and folate at a range of 700-1500 μg.

23. The method according to claim 19, wherein the condition to be treated is selected from the group consisting of obsessive-compulsive disorder (OCD); learning disabilities; school problems; tick disorders; autism; brain and spinal cord tumors; cerebral palsy; depression; epilepsy and seizure disorders; headaches; hydrocephalus; learning disorders; muscular dystrophy; multiple sclerosis; myasthenia gravis; neurofibromatosis and other neurocutaneous diseases; neurogenetic diseases; neuroimmune diseases; neurovascular disorders; scoliosis; sleep disorders; spasticity; spina bifida and myelodysplasia; spine anomalies; traumatic brain injury; tuberous sclerosis; neuromuscular diseases, metabolic degenerative diseases of the nervous system, developmental disorders of the nervous system; and conduct disorders.

24. The method according to claim 19, wherein the condition to be treated is selected from the group consisting of amyotrophic lateral sclerosis (ALS); anxiety disorders; agnosia; Alzheimer's disease; Lou Gehrig's disease; Bell's Palsy; benign essential blepharospasm; cerebral palsy; chronic inflammatory demyelinating polyneuropathy; dementia, depression, epilepsy; headache; Huntington's disease; learning disabilities; migraine, multiple sclerosis; psychiatric disorders (i.e., schizophrenia, bipolar disorder, etc.); narcolepsy; motor neuron diseases; neurofibromatosis; Parkinson's disease; peripheral neuropathy; seizure disorder; stroke; tardive dyskinesia; tremor; and post traumatic stress disorder.
